(19) United States
(12) Patent Application Publication
(10) Pub. No.: US 2009/0055994 A1
(43) Pub. Date: Mar. 5, 2009

(54) PREPARATION OF THIN-WALLED ARTICLES OF POLYURETHANEUREA

(75) Inventor: ROBERT O. WALDBAUER, JR., Waynesboro, VA (US)

Correspondence Address: INVISTA NORTH AMERICA S.A.R.L., THREE LITTLE FALLS CENTRE/1052, 2801 CENTERVILLE ROAD, WILMINGTON, DE, 19808, US

(73) Assignee: INVISTA NORTH AMERICA S.A.R.L., Wilmington, DE (US)

(21) Appl. No.: 12/198,623

(22) Filed: Aug. 26, 2008

Related U.S. Application Data
(60) Provisional application No. 60/969,040, filed on Aug. 30, 2007.

(51) Int. Cl. A41D 19/00; C08G 18/00; C08L 75/04; C08L 75/12; A61B 19/04; C08K 5/20; C08K 5/3415; B05D 3/02

(52) U.S. Cl. 2/167; 524/590; 524/233; 524/104; 427/385.5; 2/161.7; 428/35.7

(57) ABSTRACT

Certain polyether- or polyester-based polyurethaneureas in dipping solutions of organic solvent at a concentration of, for example, 12 to 20%, are particularly suited for use in a mandrel-dipping process for producing thin-walled elastic articles, such as surgical gloves. Such dipping solutions can be easily prepared, for example, by dissolving yarn comprising the polyurethaneurea and without the need to utilize metal chlorides, such as lithium chloride, in the yarn dissolving procedure.

### CROSS-REFERENCE TO RELATED APPLICATIONS

This application claims benefit of priority from Provisional Application No. 60/969,040, filed Aug. 30, 2007. This application hereby incorporates by reference Provisional Application No. 60/969,040 in its entirety.

### FIELD OF THE INVENTION

This invention relates to a process for making thin-walled elastic articles from polyether- and polyester-based polyurethaneurea solutions, to the polyurethaneurea solutions themselves, and to articles made therefrom. More particularly, the invention concerns a process which employs particular polyether-based and polyester-based polyurethaneurea solutions for making thin-walled articles, such as surgical gloves, condoms and the like, which have superior resistances to puncture and tear, compared to such articles made from conventional rubber latex.

### BACKGROUND OF THE INVENTION

Elastomeric gloves, such as those made from conventional rubber latex, are known for use in sterile, surgical, and chemical environments. Such gloves should have ease of donning, good fit, comfort, and tactility (i.e., the ability to feel objects through the gloves), low set and high resistance to tear and puncture. Conventional rubber-latex gloves are made by dipping a mandrel which is pre-coated with a coagulant into an aqueous rubber emulsion. To provide gloves with adequate strength and avoid pinholes, the dipped rubber-latex gloves typically are in the range of 0.18 to 0.20 mm thick. Such thicknesses somewhat limit the glove user's digital dexterity and tactility.

Hess et al, U.S. Pat. No. 2,814,834, and Kobayashi et al, U.S. Pat. No. 3,553,308, disclose substituting a synthetic polyesterurethaneurea for rubber latex to produce gloves or other thin-walled articles, by “reaction dipping” methods which include (a) coating a mandrel by dipping it into a solution of an isocyanate-terminated polyester prepolymer, (b) then dipping the thusly-coated mandrel into a solution of a diamine chain-extending agent to react the diamine with the isocyanate-terminated prepolymer to form a polyesterurethaneurea coating on the mandrel, (c) removing the solvent from the coating and (d) then stripping the coating from the mandrel to provide the finished article.

Woodcock et al, UK Patent Application GB 2181691, discloses a glove-making process which comprises dipping a form into a solution of a polyesterurethane having polyester segments of 1,000 to 3,000 molecular weight. The only polyesterurethane specifically exemplified by Woodcock et al is made by isocyanate capping of a polyester glycol of 2,000 molecular weight under conditions that result in an 8.7% NCO content (i.e., 8.7 weight % of unreacted isocyanate groups after the capping).

Dreibelbis et al, U.S. Pat. Nos. 5,391,343 and 5,728,340, both disclose dipping solutions of certain polyester-based and polyether-based polyurethaneurea polymers which are prepared by reacting polyurethaneurea-forming materials in situ within solvents such as N,N-dimethylacetamide. Thin-walled articles such as gloves can then be prepared by dipping a mandrel into such virgin dipping solutions, removing the mandrel from the solution, and then evaporating the solvent from the coated mandrel to thereby form the desired elastic article on the mandrel.

Polyurethaneurea solutions have also been suggested for coating fabric gloves. For example, Ishiwata, Japanese Patent Application Publication 60-033847 (1985), discloses dissolving spandex fibers, prepared from polyether glycol, 4,4′-diphenylmethane diisocyanate, and ethylene diamine, in dimethylformamide containing 0.05 to 10% lithium chloride to form a solution having 19% solids and a solution viscosity of 50 poise. Then, fabric gloves, fitted on a metal mandrel are dipped into and removed from the solution and the dimethylformamide solvent is allowed to evaporate, thereby completing the coated glove. Ishiwata notes that when lithium chloride is omitted from the solution, gel formation and attendant glove fabrication difficulties are encountered.

Notwithstanding related art technology which involves preparation of thin-walled articles from dipping solutions of polyurethaneurea materials, it would be advantageous to identify additional types of polyuretheneureas for use in novel dipping solutions that can be used in processes for preparing thin-walled articles of especially desirable properties. It would be further desirable to identify dipping solutions and article preparation processes of this type wherein the dipping solutions can be readily prepared by dissolving polyurethaneurea-based, e.g., spandex, yarns in the dipping solution solvents. Dipping solutions of this type can then be prepared at the site of article manufacture with the solution-forming yarn being shipped to that manufacturing site separately from and more easily than the solvents used.

### SUMMARY OF THE INVENTION

In one aspect, the present invention is directed to a process for fabricating a thin-walled elastic article such as a surgical glove. Such a process comprises A) preparing a very specific type of polyetherurethaneurea; B) forming a solution of the polyetherurethaneurea in an organic solvent, the polyetherurethaneurea being present in the solution at a concentration up to 25% by total solution weight, and the solution having a viscosity, measured at 25° C., in the range of 1,000 to 20,000 cP; C) optionally, degassing the thusly formed polyetherurethaneurea solution; D) dipping a mandrel into the solution; and then E) removing the mandrel therefrom to form a solution-coated mandrel; F) drying the coated mandrel; and G) removing the resulting dried thin-walled article from the mandrel.

The specific polyetherurethaneurea used in this process can be one of two types. One such type comprises the reaction product of: (a) a poly(tetramethylene-co-ethyleneether) glycol comprising constituent units derived by copolymerizing tetrahydrofuran and ethylene oxide wherein the portion of the units derived from ethylene oxide is present in the poly(tetramethylene-co-ethyleneether) glycol from greater than about 5 to about 70 mole percent and the number average molecular weight of said glycol is from about 1000 Daltons to about 4000 Daltons; (b) at least one diisocyanate; and (c) at least one diamine chain extender selected from the group consisting of ethylene diamine, 1,3-butanediamine, 1,4-butanediamine, 1,3-diamino-2,2-dimethylbutane, 1,6-hexanediamine, 1,2-propanediamine, 1,3-propanediamine, N-methylaminobis(3-propylamine), 2-methyl-1,5-pentanediamine, 1,5-diaminopentane, 1,4-cyclohexanediamine, 1,3-diamino-4-methylcyclohexane, 1,3-cyclohexane-diamine, 1,1-methylene-bis(4,4′-diaminohexane), 3-aminomethyl-3,5,5-trimethylcyclohexane, 1,3-diaminopenthane, m-xylylene diamine, hydrazine, and mixtures thereof.

The other suitable type of polyetherurethaneurea comprises the reaction product of: (a) poly(tetramethylene ether) glycol (b) at least one diisocyanate wherein the mole ratio of diisocyanate to glycol is from about 1.40 to about 2.04; and (c) at least one diamine chain extender selected from the group consisting of ethylene diamine, 1,3-butanediamine, 1,4-butanediamine, 1,3-diamino-2,2-dimethylbutane, 1,6-hexanediamine, 1,2-propanediamine, 1,3-propanediamine, N-methylaminobis(3-propylamine), 2-methyl-1,5-pentanediamine, 1,5-diaminopentane, 1,4-cyclohexanediamine, 1,3-diamino-4-methylcyclohexane, 1,3-cyclohexane-diamine, 1,1-methylene-bis(4,4′-diaminohexane), 3-aminomethyl-3,5,5-trimethylcyclohexane, 1,3-diaminopentane, m-xylylene diamine, hydrazine, and mixtures thereof; wherein the poly(tetramethylene ether) glycol is substantially free of diols having molecular weights of less than about 250 daltons. Preferably, the solution of either type of these polyetherurethaneureas is formed by dissolving yarn which has been made from and which thus comprises the polyetherurethaneurea.

In another aspect, the present invention is directed to a similar process which uses a certain type polyesterurethaneurea yarn to form the solution into which the mandrel is dipped. Such a yarn comprises polyesterurethaneurea which is a reaction product made by reacting at least one diisocyanate with a polyester diol to form an isocyanate-capped prepolymer having a % NCO in the range of 1.4 to 2.0 weight %, the polyester diol having been derived from the reaction of adipic acid with a mixture of ethylene glycol (2G) and 1,4-butanediol (4G) in the weight ratio of 2G/4G in the range of 20:80 to 80:20 and having a number average molecular weight in the range of 3,000 to 5,000; and by chain-extending the prepolymer with a diamine chain extender selected from the group consisting of ethylene diamine, 1,3-butanediamine, 1,4-butanediamine, 1,3-diamino-2,2-dimethyl-butane, 1,6-hexanediamine, 1,2-propanediamine, 1,3-propanediamine, N-methylaminobis(3-propylamine), 2-methyl-1,5-pentanediamine, 1,5-diaminopentane, 1,4-cyclohexanediamine, 1,3-diamino-4-methyl-cyclohexane, 1,3-cyclohexane-diamine, 1,1-methylene-bis(4,4′-diamino-hexane), 3-aminomethyl-3,5,5-trimethylcyclohexane, 1,3-diamino-pentane, m-xylylene diamine, hydrazine, and mixtures thereof. In this process embodiment, the polyesterurethaneurea yarn is dissolved in the solvent in the substantial absence of metal chlorides such as lithium chloride.

In other aspects, the present invention is directed to dipping solutions which comprise these several types of polyether- or polyester-based polyurethaneureas. Articles made from such dipping solutions and by such article preparation processes are also claimed.

### DETAILED DESCRIPTION OF THE INVENTION

The invention will now be described in greater detail with particular reference to the fabrication of thin-walled surgical gloves from polyetherurethaneurea and polyesterurethaneurea solutions of the invention. The term “thin-walled”, as used herein, generally refers to a thickness of no greater than about 0.5 mm, preferably no greater than about 0.18 millimeters. The term “polyurethaneurea” refers to a long chain synthetic polymer that consists essentially of alternating “soft segments” of polyether or polyester and “hard segments” derived from the reaction of an isocyanate and a diamine chain extender. The isocyanate end-group content of isocyanate-terminated prepolymer is described by the % NCO. “Molecular weight” refers to number average molecular weight. Also, several abbreviations are used herein, with the following meanings:
2G ethylene glycol
4G 1,4-butanediol
6 adipic acid
THF tetrahydrofuran
EO ethylene oxide
MDI methylene bis-(4-phenylisocyanate)
PICM methylene bis-(4-cyclohexylisocyanate)
EDA ethylenediamine
HMPD 1,3-diaminocyclohexane
1,2PDA 1,2-diaminopropane
1,3PDA 1,3-diaminopropane
MXD metaxylylene diamine
DMAc N,N-dimethylacetamide

In accordance with the present invention, polyurethaneurea solutions are prepared by dissolving polyurethaneurea in a polar aprotic solvent, such as dimethylacetamide (DMAc), dimethyl sulfoxide, N-methylpyrrolidone, dimethylformamide or the like. The solution has a falling-ball viscosity in the range of 1000 and 20000 centipoise and preferably has a polyurethaneurea concentration in the range of 12 to 20%. Solutions with viscosities greater than about 20000 centipoise tend to trap air bubbles and are difficult to evenly dip-coat onto a mandrel. Solutions with viscosities lower than about 1000 centipoise often wet the mandrel unevenly. Solutions with a polymer concentration in the range of 14 to 17% and a viscosity in the range of 5000 to 10000 centipoise are preferred. The lower ends of the preferred ranges are particularly preferred when two separate dipping and drying steps are employed to form articles with greater thickness.

When the polyurethaneurea for use in the present invention is polyether-based, the polyether segments can be one of two types. One such type comprises the THF/EO copolymer glycols. The portion of the units derived from ethylene oxide can be present in the poly(tetramethylene-co-ethyleneether) glycol from greater than about 5 to about 70 mole percent, preferably from about 37 to about 70 mole percent and the number average molecular weight of said glycol is from about 1000 Daltons to about 4000 Daltons, preferably from about 1900 to about 4000 Daltons. When such polyether segments are of this type, the hard segments comprise the reaction product of at least one diisocyanate; and at least one diamine chain extender selected from the group consisting of ethylene diamine, 1,3-butanediamine, 1,4-butanediamine, 1,3-diamino-2,2-dimethylbutane, 1,6-hexanediamine, 1,2-propanediamine, 1,3-propanediamine, N-methylaminobis(3-propylamine), 2-methyl-1,5-pentanediamine, 1,5-diaminopentane, 1,4-cyclohexanediamine, 1,3-diamino-4-methylcyclohexane, 1,3-cyclohexane-diamine, 1,1-methylene-bis(4,4′-diaminohexane), 3-aminomethyl-3,5,5-trimethylcyclohexane, 1,3-diaminopentane, m-xylylene diamine, hydrazine, and mixtures thereof. Preferably, the diamine chain extender further comprises at least one primary or secondary amine chain terminator.

In another type of polyetherurethaneurea useful herein, the polyether segments comprise poly(tetramethylene ether) glycol. These polyetherurethaneureas comprise the spandex materials described in U.S. Pat. No. 6,984,708, incorporated herein by reference.

Such materials comprise the reaction product of the homopolymer glycol with at least one diisocyanate, preferably 1-isocyanato-4-[(4-isocyanatophenyl)methyl]benzene, wherein the mole ratio of diisocyanate to glycol is from about 1.40 to about 2.04; and with at least one diamine chain extender of the type hereinbefore described. Preferably such a chain extender comprises from about 35 to about 55 mole percent ethylene diamine; and from about 45 to about 65 mole percent 1,2-propanediamine. In such materials, the poly(tetramethylene ether) glycol is substantially free of diols having molecular weights of less than about 250 Daltons. Preferred spandex materials of this type are those wherein the poly(tetramethylene ether) glycol has a number average molecular weight of from about 1600 Daltons to about 2500 Daltons; wherein the mixture of chain extenders used comprises from about 40 to about 50 mole percent ethylene diamine, and from about 50 to about 60 mole percent 1,2-propanediamine; and wherein the mixture of chain extenders further comprises at least one primary or secondary amine chain terminator.

When the polyurethaneurea for use in the invention is polyester-based, the polyurethaneurea is preferably formed from a hydroxyl-terminated adipate copolyester (2G/4G-6), which is the reaction product of adipic acid (6) and a mixture of ethylene glycol (2G) and 1,4-butanediol (4G). Typically, the weight ratio of 2G to 4G is in the range of 20:80 to 80:20, and preferably in the range 40:60 to 75:25. The hydroxyl-terminated adipate copolyester is reacted with excess diisocyanate to produce isocyanate-terminated prepolymer. Typically, the isocyanate end-group content (i.e., “% NCO”) of the prepolymer is greater than 1.4% to avoid excessive tackiness and less than 2.0% to avoid excessive load power in the final article. The isocyanate-terminated prepolymer which is then chain extended with a diamine chin extender to form the polyesterurethaneurea. To control molecular weight, a minor amount of a primary or secondary amine, such as diethylamine can be used as a chain terminator. Suitable diisocyanates include MDI, PICM and the like. MDI is preferred.

Suitable diamines include EDA, 1,2-PDA, 1,3-PDA, HMPD, MXD, mixtures thereof and the like. EDA is preferred. Suitable solvents for solutions of the invention include dimethylformamide, DMAc, dimethyl sulfoxide, and N-methylpyrrolidone. DMAc is preferred. Typically, the copolyester glycol has a molecular weight in the range of 3,000 to 6,000. Polyesterurethaneureas made from copolyesterdiol 2G/4G-6 by capping with MDI and chain extending with EDA are preferred for the best balance of properties for glove fabrication.

Various known additives can be included in the polyurethaneurea solutions of the invention and thin-walled articles made therefrom for various purposes. For example, phenolic antioxidants, such as Cyanox* 1790 (sold by American Cyanamid Co.), Santowhite Powder* 345 (sold by Monsanto Chemical Co.), the condensation polymer from p-cresol and divinyl benzene, copolymers containing tertiary amine such as DIPAM/DM (diisopropylaminoethylmethacrylate and n-decylmethacrylate in a 70/30 weight ratio), or the polyurethane formed by reaction of t-butyldiethanolamine and methylene-bis-(4-cyclohexylisocyanate) and the like. Among other additives suitable for use in the solutions and products of the invention are conventional agents such as thermal stabilizers, UV stabilizers, pigments, dyes, titanium dioxide, and the like.

To prepare thin-walled shaped articles, such as gloves, from the solutions of the invention, entrapped and/or dissolved air or other gases are first removed from the solution. Gas removal can be accomplished by applying vacuum on the solution for a few minutes. Then, a mandrel of the desired size and shape is dipped into the degassed solution, preferably at an angle of about 80 to almost 90 degrees to the vertical with the mandrel “fingers” entering first and the palm facing upward. The mandrel is kept immersed in the solution for about 5 to 30 seconds. The solution temperature is typically at about 20 to 60° C. The mandrel may be hot or at room temperature depending on desired glove thickness.

After immersion, the mandrel is slowly removed (over a period of about 10 to 120 seconds) from the solution and excess solution allowed to drain for about 1 to 5 minutes, with the mandrel fingers in a downward position. When the mandrel is removed from the solution, a web of solution forms almost immediately between the fingers of the mandrel. Usually, draining of solution from the coated mandrel is continued until the web disappears and all dripping has substantially stopped. The coated mandrel is placed, with the fingers pointing upward, in a convection oven or infrared dryer maintained at about 90° C.-150° C. for a period of about 8 to 20 minutes to remove residual solvent. Lower temperatures can be used but longer drying times are needed. After drying, the coated mandrel is allowed to cool to room temperature. Then, the glove is removed from the mandrel. The resultant glove produced by this procedure usually has a thickness of about 0.002 to 0.020 inch (0.05 to 0.5 mm). Such thin-walled gloves are particularly desired for surgical gloves and use in clean rooms.

Gloves may be double-dipped for increased thickness, as illustrated in Examples below. Draining, drying, and glove removal, etc., may be done in the usual manner. When using a double dip for increased glove thickness, the mandrel preferably is heated to about 85° C. prior to the first dip. Such preheating helps avoid non-uniformities, such as thick-thin spots, streaks, blisters, etc., in the resultant final article. Special gloves with fingers or fingers-and-palm area of extra thickness, e.g., 0.020 inch (0.50 mm) thickness, can be made with the double-dip procedure by dipping the mandrel to the appropriate depth during the first or second dip. Similar double-dipping procedures can be used to prepare gloves or other articles having multiple layers, each of the same or a different polymer.

Preferably, the mandrel has a matte surface, is shaped for optimum glove fit, and is made of aluminum or ceramic, the latter being most preferred. Usually, it is desirable to coat the mandrels with perfluoropolymers to facilitate removal of the completed article from the mandrel without damaging the article. Perfluoropolymer sprays, such as “RemGrit TLF 50”, sold by Rem Chem Division of RemGrit Corporation, of Bridgeport, Conn., are suitable. Other release agents, such as silicone oil (e.g., Dow Corning FF-400) are also satisfactory for application to the mandrels before they are dipped in the polyurethaneurea solution. Alternatively, the release agents can be mixed with the polymer solution. Anti-tack agents such as molecular sieves as taught by DiMaio in U.S. Pat. No. 6,720,049 may also be added to the dipping solutions. Removal of the article from the mandrel is also made easier by dipping the mandrel with the formed article still in place into an aqueous solution of surfactants and then removing the article from the mandrel.

A cuff can be formed on a dried glove by rolling the “sleeve” (i.e., upper wrist portion) onto itself. It is preferable to do this while the glove is still hot (about 85° C.) so that the cuff material sticks to itself without additional heat. A well-defined demarcation line at the mandrel “elbow” aids the formation of cuffs. Note that following removal of the dipped mandrel with the fingers in the down position, sufficient “drip time” should be allowed to assure that enough solution and solvent drips from the mandrel, so that when the mandrel is inverted (fingers up) for final evaporation and removal of solvent, polymer solution will not flow below the demarcation line.

Anti-tack agents, such as stearates, talc, corn starch, and the like can be applied to the inside and/or outside of the glove before packaging. Alternatively, perfluoroethylene polymers can be sprayed onto the inside and/or outside of the glove to eliminate tackiness. It is preferred to apply the perfluoroethylene polymer while the glove is still hot to improve adhesion of the perfluoroethylene particles to the surface of the glove.

Another method of preparing tack-free gloves is to dip the mandrel sequentially into different baths, each containing a different polymer solution. In a preferred version of this method, a polyesterurethaneurea layer can be formed as a middle layer between layers of polyetherurethaneurea and/or polyvinylpyrrolidone. For compatibility with the polyesterurethaneurea, DMAC is the preferred solvent for each polymer solution used to produce the layered glove.

The strength, freedom from pin-holes and general integrity of the shaped article, especially when the article is a glove, condom, or the like, is of great importance to the performance of the article. Testing for strength and freedom from leaks due to pin holes is an important quality control measure. For example, measures of these two characteristics can be made while the article is still on the mandrel or forming mold. For example, the wrist portion of a glove fan be sealed to the mandrel with an inflatable collar and then a fixed volume of air or water can be injected into the glove. A measure of the strength of the glove is indicated by the initial pressure contained by the glove; the absence of pinholes is indicated by the maintenance of a constant pressure during a predetermined time interval. After testing, the glove is deflated and removed from the mandrel. In this way, each article may be subjected to leak testing before removal from the mandrel.

### Test Procedures

Various characteristics and properties mentioned in the preceding discussion and in some instances reported in the examples below can be determined by the following methods:

Solution viscosity can be determined in accordance with the general method of ASTM D1343-69 with a Brookfield RVDVII Pro+ viscometer, equipped with a small sample adapter with SC 4 cup and #21 spindle and a circulating water bath to maintain temperature at 25±1° C.

Glycol number average molecular weight can be determined from the hydroxyl number of the polyether diol or polyester diol. Hydroxyl number is measured by the imidazole-pyridine catalyst method described in S. L. Wellon et al., “Determination of Hydroxyl Content of Polyurethane Polyols and Other Alcohols”, Analytical Chemistry, vol. 51, No. 8, pp. 1374-1376 (July 1980).

The isocyanate end-group concentration in the capped prepolymer, NCO, can be measured by the method of S. Siggia, “Quantitative Organic Analysis via Functional Group”, 3rd Edition, Wiley & Sons, New York, pages 559-561 (1963).

Resistance to deformation and elastic properties of samples can be measured in accordance with the general method of ASTM D273 1-72, except that the thread of the ASTM method is replaced by a sample of cast film of ⅛-inch (0.32-cm) width, 2-inch (5-cm) length and a measured thickness. Denier of the film is determined from the weight of a known length of a 0.32-cm-wide strip of the film. The samples are subjected to five 0 to 300% extension/retraction cycles at a constant elongation rate of 800% per minute. Load power is determined in milligrams per original denier and reported in deciNewtons per Tex for LP₁₀₀ and LP₂₀₀, by measuring the load on the sample during the first cycle as the sample is extended by 100% and 200%, respectively. After completion of the fifth cycle the film strip is relaxed for 30 seconds and its increase in length is determined as a percent of its unstretched original length and reported as percent set.

Tear strength of the film samples can be determined in pounds force per inch of sample thickness by the general procedure of ASTM D470-82 and is reported in the Examples in Newtons per centimeter.

Puncture resistance can be measured in accordance with a procedure in which a sample of film is held in a 3-½-inch-diameter (8.9-cm) circular holder in a flat, horizontal position and is then is penetrated by a vertical probe fitted with an sharp blade (i.e., a No. 10 Exacto knife blade) attached to the crosshead of an Instron testing machine, with the crosshead moving at a vertical rate of two inches per minute (5.1 cm/min). Puncture resistance is determined in pounds force per inch of sample thickness and is reported in the Examples in Newtons per cm.

The practical significance of the above described measurements with regard to typical articles of the invention (e.g., surgical gloves) is as follows. Load power at extensions of 100% and 200% represent the retractive force in a film or glove as it is stretched. An extension of 200% approximates the maximum stretch experienced when a glove is pulled on or removed. Because a glove must return to its original shape after being pulled on, a low value of no greater than 15% set is desired. High resistance to tear is needed for an article, such as a glove, to survive repeated pulling on and taking off. Puncture resistance is also highly desirable.

### EXAMPLES

The invention is further illustrated by the following examples of several invention embodiments. These examples are included for purposes of illustration and are not intended to limit the scope of the invention, which scope is defined by the appended claims. The reported results are believed to be representative but do not constitute all the runs involving the indicated materials.

### Example 1

This example illustrates preparation of a glove dipping solution made by dissolving yarn. Spandex yarn was made from a prepolymer obtained from the reaction of MDI and 2000 molecular weight poly(tetramethylene ether) glycol capped with MDI to 2.40% NCO content. This prepolymer was chain extended with a mixture of 55 mol % 1,2 propanediamine and 45 mol % ethylenediamine and terminated with diethylamine. The yarn was removed from the yarn cake in layers about 0.5 cm thick. The layers were cut into lengths of approximately 2.5 cm.

Following the addition of DMAc (2430 g) to a 1 gallon jar, 570 g of the cut yarn was added to the jar. The jar was then placed in an oven at 45° C. for 12 hours to solvate the yarn. No metal chlorides such as lithium chloride were present in the jar during yarn dissolution. After 12 hours, the mixture was stirred under nitrogen with stir rod capped with a 3 cm disk for 30 minutes. The viscosity of the resulting glove dipping solution was approximately 13,000 cP.

### Example 2

This example illustrates preparation of another glove dipping solution made by dissolving yarn. Spandex yarn was made from a prepolymer obtained from the reaction of MDI and a 2500 molecular weight copolyether glycol made from 38% ethylene oxide and 62% terathydrofuran. The % NCO of this prepolymer was 2.0%. The prepolymer was extended with ethylene diamine and molecular weight was controlled by diethylamine. The intrinsic viscosity of the yarn was 1.3 dl/g. The yarn was removed from the yarn cake in layers about 0.5 cm thick. The layers cut into lengths of approximately 2.5 cm. (Suitable yarn can also be made from a polyesterurethaneurea which comprises 2G/4G polyester glycol capped with MDI to a 1.80% NCO, extended with 100% EDA, with a cyclohexylamine terminator.)

Following the addition of DMAc (2475 g) to a 1 gallon jar, 525 g of the cut yarn was added to the jar. The jar was then placed in an oven at 45° C. for 12 hrs to solvate the yarn. Again, no metal chlorides such as lithium chloride were present in the jar during yarn dissolution. After 12 hours, the mixture was stirred under nitrogen with a stir rod capped with a 3 cm disk for 30 minutes. The viscosity of the resulting glove dipping solution was approximately 13,000 cP.

### Example 3

This example illustrates preparation of gloves using dipping solutions such as those of Examples 1 and 2. Such dipping solutions are used in a DipTech Systems Diplomat automated dipping apparatus. For each such solution, a glove is made as follows:

A ceramic mandrel was heated to 110° C. in a Despatch Industries forced air oven and then vertically attached to the automated dipping apparatus. While still hot, the mandrel was lowered into the solution at 1 inch per second at an angle of 14.5 degrees from vertical until the mandrel was covered approximately 3 inches past the wrist at which time the form was held stationary for 11 seconds.

The mandrel was moved to 3.9 degrees from vertical and then withdrawn at a rate of 0.025 inches per second until the tips of the fingers were completely removed from the solution. At this point, the mandrel was moved to a vertical position and then further elevated at a rate of 2 inches per second to a height of approximately 9 inches above the tank and held at this height for 60 second to allow excess solution to drip down from the fingers. Using an automated rotation program, the mandrel was rotated 360° through the x-y plane at rate of 10 rpm and on the z-axis at 15 rpm for approximately 4 minutes to ensure the solution was uniformly distributed on the mandrel.

After the rotation program was completed, the mandrel was placed in a Despatch Industries forced air oven in a vertical position with the finger pointing up and with the oven maintained at 110° C. for a period of 12 minutes to evaporate the DMAc. The dry mandrel was placed in a bucket of water to cool the mandrel and facilitate removal of the glove.

What is claimed is:

1. A process for fabricating a thin-walled elastic article, which process comprises:
A) preparing a polyetherurethaneurea selected from the group consisting of
i) the reaction product of: (a) a poly(tetramethylene-co-ethyleneether) glycol comprising constituent units derived by copolymerizing tetrahydrofuran and ethylene oxide wherein the portion of the units derived from ethylene oxide is present in the poly(tetramethylene-co-ethyleneether) glycol from greater than about 5 to about 70 mole percent and the number average molecular weight of said glycol is from about 1000 Daltons to about 4000 Daltons; (b) at least one diisocyanate; and (c) at least one diamine chain extender selected from the group consisting of ethylene diamine, 1,3-butanediamine, 1,4-butanediamine, 1,3-diamino-2,2-dimethylbutane, 1,6-hexanediamine, 1,2-propanediamine, 1,3-propanediamine, N-methylaminobis(3-propylamine), 2-methyl-1,5-pentanediamine, 1,5-diaminopentane, 1,4-cyclohexanediamine, 1,3-diamino-4-methylcyclohexane, 1,3-cyclohexane-diamine, 1,1-methylene-bis(4,4′-diaminohexane), 3-aminomethyl-3,5,5-trimethylcyclohexane, 1,3-diaminopentane, m-xylylene diamine, hydrazine, and mixtures thereof; and
ii) the reaction product of: (a) poly(tetramethylene ether) glycol (b) at least one diisocyanate wherein the mole ratio of diisocyanate to glycol is from about 1.40 to about 2.04; and (c) at least one diamine chain extender selected from the group consisting of ethylene diamine, 1,3-butanediamine, 1,4-butanediamine, 1,3-diamino-2,2-dimethylbutane, 1,6-hexanediamine, 1,2-propanediamine, 1,3-propanediamine, N-methylaminobis(3-propylamine), 2-methyl-1,5-pentanediamine, 1,5-diaminopentane, 1,4-cyclohexanediamine, 1,3-diamino-4-methylcyclohexane, 1,3-cyclohexane-diamine, 1,1-methylene-bis(4,4′-diaminohexane), 3-aminomethyl-3,5,5-trimethylcyclohexane, 1,3-diaminopentane, m-xylylene diamine, hydrazine, and mixtures thereof; wherein the poly(tetramethylene ether) glycol is substantially free of diols having molecular weights of less than about 250 Daltons;
B) forming a solution of the polyetherurethaneurea in an organic solvent, the polyetherurethaneurea being present in the solution at a concentration up to 25% by total solution weight, and the solution having a viscosity, measured at 25° C., in the range of 1,000 to 20,000 cP;
C) optionally, degassing the thusly formed polyetherurethaneurea solution;
D) dipping a mandrel into the solution; and then
E) removing the mandrel therefrom to form a solution-coated mandrel;
F) drying the coated mandrel; and
G) removing the resulting dried thin-walled article from the mandrel.

2. A process according to claim 1 wherein said polyetherurethaneurea is fashioned into yarn material and said solution has been formed by dissolving, in the substantial absence of metal chlorides, the yarn material which comprises said polyetherurethaneurea.

3. A process according to claim 1 wherein the at least one diisocyanate used to form said polyetherurethaneurea comprises 1-isocyanato-4-[(4-isocyanatophenyl)methyl]benzene.

4. A process according to claim 1 wherein the at least one diamine chain extender used to form said polyetherurethaneurea is selected from the group consisting of ethylene diamine and mixtures comprising from about 35 to about 55 mole percent ethylene diamine and from about 45 to about 65 mole percent 1,2-propanediamine.

5. A process according to claim 1 wherein, in reaction product i), the ethylene oxide is present in the glycol reactant to the extent of from about 37 to about 70 mole percent and the molecular weight of the glycol reactant ranges from about 1600 to about 4000 Daltons.

6. A process according to claim 1 wherein said polyetherurethaneurea is present in said solution at a concentration of from about 12% to about 20% by weight of the solution.

7. A process according to claim 1 wherein the polyether used to form said polyetherurethaneurea is poly(tetramethylene ether) glycol having a number average molecular weight of from about 1900 Daltons to about 2500 Daltons.

8. A process according to claim 1 wherein, in reaction product ii), a mixture of chain extenders is used comprising from about 40 to about 50 mole percent ethylene diamine, and from about 50 to about 60 mole percent 1,2-propanediamine.

9. A process according to claim 1 wherein said at least one diamine chain extender further comprises at least one primary or secondary amine chain terminator.

10. A process for fabricating a thin-walled elastic article, which process comprises:
A) preparing a yarn comprising a polyesterurethaneurea which comprises a reaction product made by reacting at least one diisocyanate with a polyester diol to form an isocyanate-capped prepolymer having a % NCO in the range of 1.4 to 2.0 weight %, the polyester diol having been derived from the reaction of adipic acid with a mixture of ethylene glycol (2G) and 1,4-butanediol (4G) in the weight ratio of 2G/4G in the range of 20:80 to 80:20 and having a number average molecular weight in the range of 3,000 to 5,000; and by chain-extending the prepolymer with at least one diamine chain extender selected from the group consisting of ethylene diamine, 1,3-butanediamine, 1,4-butanediamine, 1,3-diamino-2,2-dimethylbutane, 1,6-hexanediamine, 1,2-propanediamine, 1,3-propanediamine, N-methylaminobis(3-propylamine), 2-methyl-1,5-pentanediamine, 1,5-diaminopentane, 1,4-cyclohexanediamine, 1,3-diamino-4-methylcyclohexane, 1,3-cyclohexane-diamine, 1,1-methylene-bis(4,4′-diaminohexane), 3-aminomethyl-3,5,5-trimethylcyclohexane, 1,3-diaminopentane, m-xylylene diamine, hydrazine, and mixtures thereof;
B) dissolving said yarn in an organic solvent in the substantial absence of metal chlorides to thereby form a solution containing polyesterurethaneurea at a concentration in the range of 12% to 20% by total solution weight, with said solution having a viscosity, measured at 25° C., in the range of 1,000 to 20,000 cP;
C) optionally, degassing the thusly formed polyesterurethaneurea solution;
D) dipping a mandrel into the solution; and then
E) removing the mandrel therefrom to form a solution-coated mandrel;
F) drying the coated mandrel; and
G) removing the resulting dried thin-walled article from the mandrel.

11. A process according to claim 10 wherein the polyesterurethaneurea is present in said solution at a concentration of from about 12% to about 20% by weight of the solution.

12. A process according to claim 10 wherein, in the polyesterurethaneurea, the weight ratio of the ethylene glycol to 1,4-butylene glycol is in the range of 40:60 to 75:25; the at least one diisocyanate comprises 1-isocyanato-4-[(4-isocyanatophenyl)methyl]benzene; and the chain-extending diamine is 1,3-diaminocyclohexane or ethylene diamine.

13. A process according to claim 10 wherein said at least one diamine chain extender further comprises at least one primary or secondary amine chain terminator.

14. A thin-walled elastic article prepared by a process according to claim 1 wherein the article is a glove having a thickness in the range of 0.05 to 0.05 mm.

15. A thin-walled elastic article prepared by a process according to claim 10 wherein the article is a glove having a thickness in the range of 0.05 to 0.5 mm.

16. A dipping solution useful for the preparation of thin-walled articles around a mandrel which has been dipped therein, which dipping solution comprises:
A) up to about 25% by weight of a polyetherurethaneurea selected from the group consisting of
i) the reaction product of: (a) a poly(tetramethylene-co-ethyleneether) glycol comprising constituent units derived by copolymerizing tetrahydrofuran and ethylene oxide wherein the portion of the units derived from ethylene oxide is present in the poly(tetramethylene-co-ethyleneether) glycol from greater than about 5 to about 70 mole percent and the number average molecular weight of said glycol is from about 1000 Daltons to about 4000 Daltons; (b) at least one diisocyanate; and (c) at least one diamine chain extender selected from the group consisting of ethylene diamine, 1,3-butanediamine, 1,4-butanediamine, 1,3-diamino-2,2-dimethylbutane, 1,6-hexanediamine, 1,2-propanediamine, 1,3-propanediamine, N-methylaminobis(3-propylamine), 2-methyl-1,5-pentanediamine, 1,5-diaminopentane, 1,4-cyclohexanediamine, 1,3-diamino-4-methylcyclohexane, 1,3-cyclohexane-diamine, 1,1-methylene-bis(4,4′-diaminohexane), 3-aminomethyl-3,5,5-trimethylcyclohexane, 1,3-diaminopentane, m-xylylene diamine, hydrazine, and mixtures thereof; and
ii) the reaction product of: (a) poly(tetramethylene ether) glycol (b) at least one diisocyanate wherein the mole ratio of diisocyanate to glycol is from about 1.40 to about 2.04; and (c) at least one diamine chain extender selected from the group consisting of ethylene diamine, 1,3-butanediamine, 1,4-butanediamine, 1,3-diamino-2,2-dimethylbutane, 1,6-hexanediamine, 1,2-propanediamine, 1,3-propanediamine, N-methylaminobis(3-propylamine), 2-methyl-1,5-pentanediamine, 1,5-diaminopentane, 1,4-cyclohexanediamine, 1,3-diamino-4-methylcyclohexane, 1,3-cyclohexane-diamine, 1,1-methylene-bis(4,4′-diaminohexane), 3-aminomethyl-3,5,5-trimethylcyclohexane, 1,3-diaminopentane, m-xylylene diamine, hydrazine, and mixtures thereof; wherein the poly(tetramethylene ether) glycol is substantially free of diols having molecular weights of less than about 250 Daltons; and
B) greater than 75% by weight of an organic solvent for said polyetherurethaneurea;
wherein said dipping solution has a viscosity of from about 1000 to 20,000 cP at 25° C.

17. A dipping solution according to claim 16 wherein the at least one diisocyanate used to form said polyetherurethaneurea comprises 1-isocyanato-4-[(4-isocyanatophenyl)methyl]benzene.

18. A dipping solution according to claim 16 wherein the at least one diamine chain extender used to form said polyetherurethaneurea is selected from the group consisting of ethylene diamine and mixtures comprising from about 35 to about 55 mole percent ethylene diamine and from about 45 to about 65 mole percent 1,2-propanediamine.

19. A dipping solution according to claim 16 wherein, in reaction product i), the ethylene oxide is present in the glycol reactant to the extent of from about 37 to about 70 mole percent and the molecular weight of the glycol reactant ranges from about 1600 to about 4000 Daltons.

20. A dipping solution according to claim 16 wherein said polyetherurethaneurea is present in said solution at a concentration of from about 12% to about 20% by weight of the solution.

21. A dipping solution according to claim 16 wherein the polyether used to form said polyetherurethaneurea is poly(tetramethylene ether) glycol having a number average molecular weight of from about 1900 Daltons to about 2500 Daltons.

22. A dipping solution according to claim 16 wherein, in reaction product ii), a mixture of chain extenders is used comprising from about 40 to about 50 mole percent ethylene diamine, and from about 50 to about 60 mole percent 1,2-propanediamine.

23. A dipping solution according to claim 16 wherein said at least one diamine chain extender further comprises at least one primary or secondary amine chain terminator.

24. A dipping solution according to claim 16 wherein said solution has been formed by dissolving, in the substantial absence of metal chlorides, yarn which comprises the polyetherurethaneurea.

25. A dipping solution according to claim 16 which has been formed by dissolving yarn segments in solvent while said yarn segments and solvent are agitated and heated to a temperature of form 40° C. to 50° C.

26. A dipping solution according to claim 16 wherein said solvent is selected from the group consisting of N,N-dimethylacetamide, dimethylformamide, N-methylpyrrolidone and mixtures thereof.

27. A dipping solution useful for the preparation of thin-walled articles around a mandrel which has been dipped therein, which dipping solution comprises:
A) up to about 25% by weight of a polyesterurethaneurea which comprises a reaction product made by reacting at least one diisocyanate with a polyester diol to form an isocyanate-capped prepolymer having a % NCO in the range of 1.4 to 2.0 weight %, the polyester diol having been derived from the reaction of adipic acid with a mixture of ethylene glycol (2G) and 1,4-butanediol (4G) in the weight ratio of 2G/4G in the range of 20:80 to 80:20 and having a number average molecular weight in the range of 3,000 to 5,000; and by chain-extending the prepolymer with at least one diamine chain extender selected from the group consisting of ethylene diamine, 1,3-butanediamine, 1,4-butanediamine, 1,3-diamino-2,2-dimethylbutane, 1,6-hexanediamine, 1,2-propanediamine, 1,3-propanediamine, N-methylaminobis(3-propylamine), 2-methyl-1,5-pentanediamine, 1,5-diaminopentane, 1,4-cyclohexanediamine, 1,3-diamino-4-methylcyclohexane, 1,3-cyclohexane-diamine, 1,1-methylene-bis(4,4′-diaminohexane), 3-aminomethyl-3,5,5-trimethylcyclohexane, 1,3-diaminopentane, m-xylylene diamine, hydrazine, and mixtures thereof; and
B) greater than 75% by weight of an organic solvent for said polyesterurethaneurea;
wherein said dipping solution has a viscosity of from about 1000 to 20,000 cP at 25° C., and
wherein said solution has been formed by dissolving, in the substantial absence of metal chlorides, yarn which comprises the polyesterurethaneurea.

28. A dipping solution according to claim 27 wherein the polyesterurethaneurea is present in said solution at a concentration of from about 12% to about 20% by weight of the solution.

29. A dipping solution according to claim 27 wherein, in the polyesterurethaneurea, the weight ratio of the ethylene glycol to 1,4-butylene glycol is in the range of 40:60 to 75:25; the at least one diisocyanate comprises 1-isocyanato-4-[(4-isocyanatophenyl)methyl]benzene; and the chain-extending diamine is 1,3-diaminocyclohexane or ethylene diamine.

30. A dipping solution according to claim 27 wherein said at least one diamine chain extender further comprises at least one primary or secondary amine chain terminator.

31. A dipping solution according to claim 27 which has been formed by dissolving yarn segments in solvent while said yarn segments and solvent are agitated and heated to a temperature of form 40° C. to 50° C.

32. A dipping solution according to claim 27 wherein said solvent is selected from the group consisting of N,N-dimethylacetamide, dimethylformamide, N-methylpyrrolidone and mixtures thereof.
